(19) United States
(12) Patent Application Publication
(10) Pub. No.: US 2012/0184504 A1
(43) Pub. Date: Jul. 19, 2012

(54) "The Use of a PAR-1 Antagonist in Combination with a P2Y12 ADP Receptor Antagonist for Inhibition of Thrombosis"

(76) Inventors: John T. Strony, Lebanon, NJ (US); Madhu Chintala, Colts Neck, NJ (US)

(21) Appl. No.: 13/498,742

(22) PCT Filed: Sep. 24, 2010
(86) PCT No.: PCT/US10/50112

§ 371 Date: Mar. 28, 2012

Related U.S. Application Data
(60) Provisional application No. 61/248,230, filed on Oct. 2, 2009.

(51) Int. Cl. A61K 31/7076; A61K 31/519; A61K 31/517; A61P 9/10; A61P 9/00; A61K 31/443; A61K 31/444; A61P 7/02; A61K 31/4365; A61K 31/5377

(52) U.S. Cl. 514/47; 514/301; 514/261.1; 514/266.24; 514/235.2

(57) ABSTRACT

The treatment and prevention of thrombotic events are provided through co-administration of PAR-1 and the P2Y12 ADP receptor antagonists. Combined inhibition of the PAR-1 and the P2Y12 ADP platelet activation pathways had synergistic antithrombotic and antiplatelet effects, as demonstrated in co-administration of SCH 602539 and cangrelor.

### RELATED APPLICATIONS

This application claims priority to application No. 61/248,230, filed on Oct. 2, 2009, the entirety of which application is incorporated herein.

### FIELD OF THE INVENTION

The present invention relates to the treatment of thrombosis.

### BACKGROUND

Atherothrombotic disease is associated with considerable morbidity and mortality. (Rosamond W, Flegal K, Furie K, Go A, Greenlund K, Haase N, Hailpern S M, Ho M, Howard V, Kissela B, Kittner S, Lloyd-Jones D, McDermott M, Meigs J, May C, Nichol G, O'Donnell C, Roger V, Sorlie P, Steinberger J, Thorn T, Wilson M, Hong Y; American Heart Association Statistics Committee and Stroke Statistics Subcommittee. Heart disease and stroke statistics—2008 update: a report from the American Heart Association Statistics Committee and Stroke Statistics Subcommittee. Circulation. 2008; 117:e25-e146). Whereas the benefits of dual antiplatelet therapy with aspirin and a P2Y₁₂ adenosine diphosphate (“ADP”) receptor antagonist have been demonstrated in a broad range of patients with atherothrombotic disease, many of these patients continue to have recurrent ischemic events. (Wiviott S D, Braunwald E, McCabe C H, Montalescot G, Ruzyllo W, Gottlieb S, Neumann F J, Ardissino D, De Servi S, Murphy S A, Riesmeyer J, Weerakkody G, Gibson C M, Antman E M; TRITON-TIMI 38 Investigators. Prasugrel versus clopidogrel in patients with acute coronary syndromes. N Engl J Med. 2007; 357:2001-2015. Yusuf S, Zhao F, Mehta S R, Chrolavicius S, Tognoni G, Fox K K; Clopidogrel in Unstable Angina to Prevent Recurrent Events Trial Investigators. Effects of clopidogrel in addition to aspirin in patients with acute coronary syndromes without ST-segment elevation. N Engl J Med. 2001; 345:494-502). This high residual risk can be attributed to the fact that aspirin and P2Y₁₂ ADP receptor antagonists (such as clopidogrel and prasugrel) only have a partial inhibitory effect on platelet-mediated thrombosis, because they each target only one of many platelet activation pathways. (Davi G, Patrono C. Platelet activation and atherothrombosis. N Engl J Med. 2007; 357:2482-2494. Meadows T A, Bhatt D L. Clinical aspects of platelet inhibitors and thrombus formation. Circ Res. 2007; 100:1261-1275). As a result, thrombosis mediated by other platelet activation pathways, including stimulation of protease-activated receptor (PAR)-1 by thrombin, continues to occur even in the presence of aspirin and a P2Y₁₂ ADP receptor antagonist, leading to ischemic events.

Thrombin is the most potent platelet agonist, as it stimulates platelet activation at very low, subnanomolar concentrations. (Brummel K E, Paradis S G, Butenas S, Mann K G. Thrombin functions during tissue factor-induced blood coagulation. Blood. 2002; 100:148-152. Mann K G. Thrombin formation. Chest. 2003; 124:4 S-10S). PAR-1 is the principal receptor for thrombin on human platelets, whereas the secondary PAR-4 receptor may contribute to platelet activation at high concentrations of thrombin. (Davi G, Patrono C. Platelet activation and atherothrombosis. N Engl J Med. 2007; 357:2482-2494. Coughlin S R. Protease-activated receptors in hemostasis, thrombosis and vascular biology. J Thromb Haemost. 2005; 3:1800-1814). Because the PAR-1 pathway is a key contributor to platelet-mediated thrombosis, PAR-1 is a valid therapeutic target for development of novel antiplatelet agents. (Davi G, Patrono C. Platelet activation and atherothrombosis. N Engl J Med. 2007; 357:2482-2494. Coughlin S R. Protease-activated receptors in hemostasis, thrombosis and vascular biology. J Thromb Haemost. 2005; 3:1800-1814). Previous preclinical studies with PAR-1 antagonists have demonstrated antithrombotic activity without an effect on bleeding time or coagulation parameters, (Cook J J, Sitko G R, Bednar B, Condra C, Mellott M J, Feng D M, Nutt R F, Shafer J A, Gould R J, Connolly T M. An antibody against the exosite of the cloned thrombin receptor inhibits experimental arterial thrombosis in the African green monkey. Circulation. 1995; 91:2961-2971. Derian C K, Damiano B P, Addo M F, Darrow A L, D'Andrea M R, Nedelman M, Zhang H C, Maryanoff B E, Andrade-Gordon P. Blockade of the thrombin receptor protease-activated receptor-1 with a small-molecule antagonist prevents thrombus formation and vascular occlusion in nonhuman primates. J Pharmacol Exp Ther. 2003; 304:855-861), supporting the clinical potential of this therapeutic approach.

SCH 530348 is a PAR-1 antagonist currently in development for the treatment of acute coronary syndrome and secondary prevention of cardiovascular events. SCH 602539 is an analog of SCH 530348. The structural formulae of the two compounds are as follows:

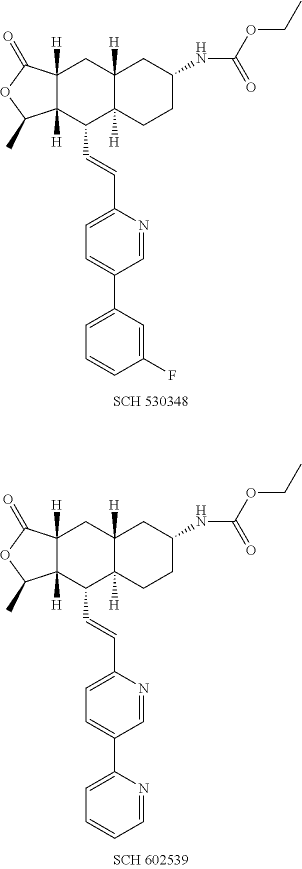

SCH 590709 is a bicyclic analog of SCH 530348 that is also a selective PAR-1 antagonist:

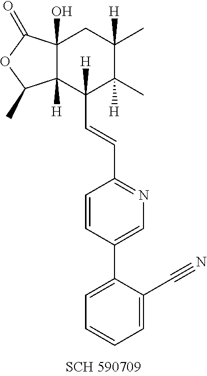

Both SCH 530348 and SCH 602539 are disclosed in U.S. Pat. No. 7,304,078. SCh 590709 is disclosed generically in U.S. Pat. No. 6,645,987, and specifically in U.S. Pat. No. 7,488,742. Crystalline forms of the bisulfate salt of SCH 530348 are disclosed in U.S. Pat. No. 7,235,567, formulations of SCH 530348 are disclosed in U.S. Ser. Nos. 11/771,571; 11/960,320; 11/771,520; and 11/860,165; methods of treating a variety conditions are disclosed in 10/705,282; 60/753,246; 11/642,505; 11/642,487; and 61/112,080; Combinations of thrombin receptor antagonists with a variety of other cardiovascular agents, including ADP antagonists are disclosed in WO2007/117621, all of which are herein incorporated in their entirety.

The clinical potential of PAR-1 antagonists as a novel class of oral, direct-acting antiplatelet agents for management of atherothrombosis is supported by the results of two recent phase 2 trials with the PAR-1 antagonist SCH 530348, which showed strong trends towards reduced incidence of ischemic events without an accompanying increase in bleeding. (Becker R C, Moliterno D J, Jennings L K, Pieper K S, Pei J, Niederman A, Ziada K M, Berman G, Strony J, Joseph D, Mahaffey K W, Van de Werf F, Veltri E, Harrington R A; TRA-PCI Investigators. Safety and tolerability of SCH 530348 in patients undergoing non-urgent percutaneous coronary intervention: a randomised, double-blind, placebo-controlled phase II study. Lancet. 2009; 373:919-928. Goto S, Yamaguchi T, Ikeda Y, Yamaguchi H, Shimizu K, Jensen P. Phase II trial of the novel antiplatelet agent, SCH 530348, in Japanese patients with non-ST segment elevation acute coronary syndromes (NSTE ACS). Eur Heart J. 2008; 29 (abstr suppl):829. Abstract P4767). Two large ongoing trials (Clinicaltrials.gov identifiers: NCT00526474 and NCT00527943) are investigating the clinical efficacy and safety of SCH 530348 in combination with standard antiplatelet therapy among patients presenting with an acute coronary syndrome and in those with a history of a prior coronary artery, cerebrovascular, or peripheral artery disease.

Cangrelor is a nonthienopyridine direct-acting P2Y12 antagonist under development for the treatment of acute coronary syndrome and as an ultrafast-acting intravenous antithrombotic agent.

### SUMMARY OF THE INVENTION

The present invention is directed to methods of treating or preventing a cardiovascular condition comprising administering to a patient in need of such treatment or prevention therapeutically effective amounts of a thrombin receptor antagonist selective for PAR-1 and of a P2Y₁₂ ADP receptor antagonist.

In some embodiments, the thrombin receptor antagonist is a compound having the chemical formula:

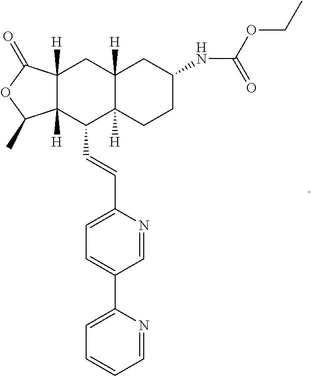

In further embodiments, the thrombin receptor antagonist is a compound having the chemical formula:

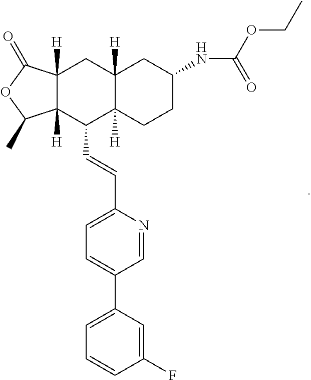

In still further embodiments, the thrombin receptor antagonist is a compound having the chemical formula:

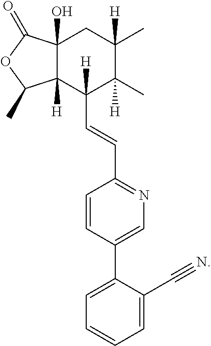

In yet further embodiments, the thrombin receptor antagonist is a compound having the chemical formula:

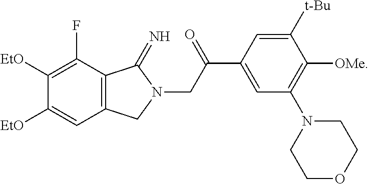

In some embodiments, the P2Y₁₂ADP receptor antagonist is a cangrelor.

In further embodiments, the P2Y₁₂ADP receptor antagonist is selected from the group consisting of ticlopidine, clopidogrel, AZD6140, ARC109318, and PRT060128.

In some embodiments, the cardiovascular condition is selected from the group consisting of acute coronary syndrome, thrombosis, stroke, myocardial infarction, peripheral arterial disease, thrombotic events in patients who have undergone percutaneous coronary intervention or cardiopulmonary bypass surgery (“CPB”), including coronary artery bypass surgery (“CABG”), cardiac valvular repair and replacement surgery, pericardial and aortic repair surgery.

In some embodiments, the prevention is secondary prevention.

In some embodiments, the thrombin receptor antagonist, the P2Y₁₂ADP receptor antagonist, and the amounts of each are selected to generate a synergistic effect in the patient.

The present invention is further directed to pharmaceutical compositions comprising therapeutically effective amounts of a thrombin receptor antagonist selective for PAR-1 and of a P2Y₁₂ ADP receptor antagonist, wherein the thrombin receptor antagonist, the P2Y₁₂ ADP receptor antagonist, and the amounts of each are selected to generate a synergistic effect in the patient.

In some embodiments, the thrombin receptor antagonist is a compound selected from those having the chemical formulae:

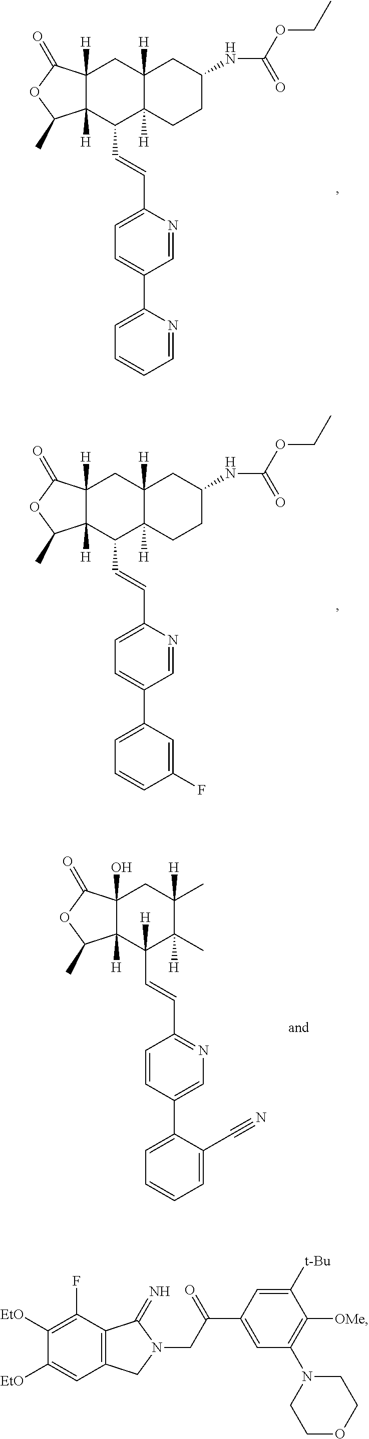

and the P2Y₁₂ADP receptor antagonist is selected from the group consisting of cangrelor, ticlopidine, clopidogrel, AZD6140, ARC109318, and PRT060128.

The present invention is further directed to kits comprising a first pharmaceutical composition comprising a therapeutically effective amount of a thrombin receptor antagonist selective for PAR-1 and a second pharmaceutical composition comprising a therapeutically effective amount of a P2Y₁₂ADP receptor antagonist, wherein the thrombin receptor antagonist, the P2Y₁₂ADP receptor antagonist, and the amounts of each are selected to generate a synergistic effect in the patient.

In some embodiments, the thrombin receptor antagonist is a compound selected from those having the chemical formulae:

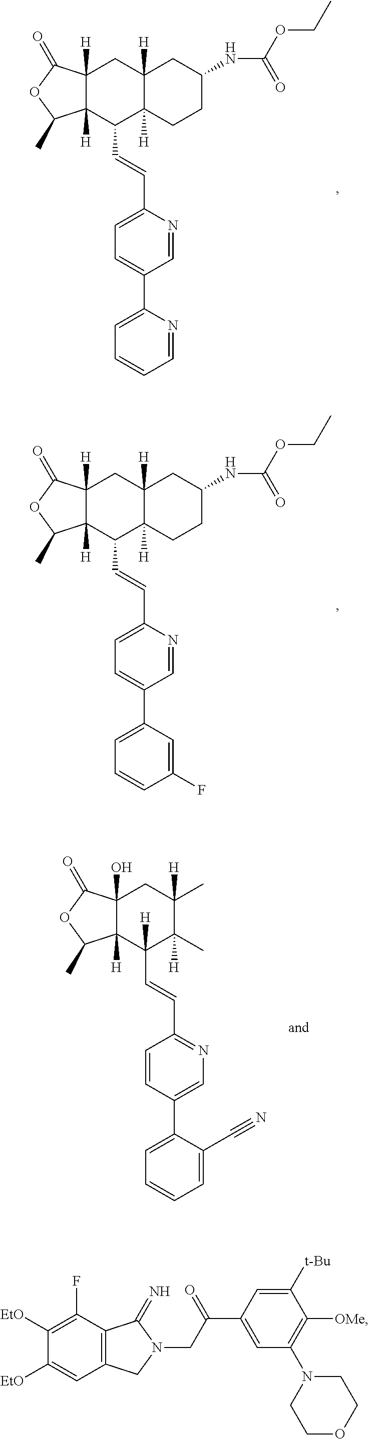

and wherein the P2Y₁₂ADP receptor antagonist is selected from the group consisting of cangrelor, ticlopidine, clopidogrel, AZD6140, ARC109318, and PRT060128.

In the above embodiments, both the thrombin receptor antagonist and the P2Y₁₂ ADP receptor antagonist are independently in the form of the free base, or of a pharmaceutically acceptable salt.

### DESCRIPTION OF FIGURES

FIG. 1 displays the observed inhibitory effects of co-administration of various doses of SCH 602539 and cangrelor on cyclic flow reductions (“CFRs”) averaged over the 2-hour monitoring period in a Folts model of thrombosis in anesthetized cynomolgus monkeys.

FIG. 2 displays the observed vs calculated inhibitory effects of co-administration of various doses of SCH 602539 and cangrelor vs single doses of SCH 602539 alone and cangrelor alone on CFRs averaged over the 2-hour monitoring period in a Folts model of thrombosis in anesthetized cynomolgus monkeys.

### DETAILED DESCRIPTION

The present invention is directed to the co-administration of a thrombin receptor antagonist (“TRA”) selective for PAR-1 and a P2Y₁₂ Adenosine-5′-diphosphate (“ADP”) receptor antagonist for the treatment of thrombosis. A study was designed to evaluate the antithrombotic efficacy of SCH 602539, a thrombin receptor antagonist selective for PAR-1; cangrelor, the most potent P2Y₁₂ ADP receptor antagonist; and their combination in a Folts model of coronary thrombosis. (Folts J. An in vivo model of experimental arterial stenosis, intimal damage, and periodic thrombosis. Circulation. 1991; 83(Suppl):IV3-IV14). Both agents can be administered parenterally and titrated.

Given the existence of species differences in the platelet thrombin receptor, (Coughlin S R. Protease-activated receptors in hemostasis, thrombosis and vascular biology. J Thromb Haemost. 2005; 3:1800-1814), cynomolgus monkeys were used, since they have the same distribution of thrombin receptors (PAR-1 and PAR-4) on their platelets as do humans. (Derian C K, Damiano B P, Addo M F, Darrow A L, D'Andrea M R, Nedelman M, Zhang H C, Maryanoff B E, Andrade-Gordon P. Blockade of the thrombin receptor protease-activated receptor-1 with a small-molecule antagonist prevents thrombus formation and vascular occlusion in nonhuman primates. J Pharmacol Exp Ther. 2003; 304:855-861). Animal experiments were conducted in accordance with the National Institutes of Health Guide for the Care and Use of Laboratory Animals and the Animal Welfare Act in a program accredited by the American Association for Accreditation of Laboratory Animal Care.

The procedures used in this study are similar to the acute artery occlusion model developed by Folts and others. (Cook J J, Sitko G R, Bednar B, Condra C, Mellott M J, Feng D M, Nutt R F, Shafer J A, Gould R J, Connolly T M. An antibody against the exosite of the cloned thrombin receptor inhibits experimental arterial thrombosis in the African green monkey. Circulation. 1995; 91:2961-2971. Folts J. An in vivo model of experimental arterial stenosis, intimal damage, and periodic thrombosis. Circulation. 1991; 83(Suppl):IV3-IV14. Mousa S A, DeGrado W F, Mu D X, Kapil R P, Lucchesi B R, Reilly T M. Oral antiplatelet, antithrombotic efficacy of DMP 728, a novel platelet GPIIb/IIIa antagonist. Circulation. 1996; 93:537-543. Wu D, Vanhoorelbeke K, Cauwenberghs N, Meiring M, Depraetere H, Kotze H F, Deckmyn H. Inhibition of the von Willebrand (VWF)-collagen interaction by an antihuman VWF monoclonal antibody results in abolition of in vivo arterial platelet thrombus formation in baboons. Blood. 2002; 99:3623-3628).

Cynomolgus monkeys were sedated with ketamine HCl (10 mg/kg, IM) followed by anesthetization with sodium pentobarbital 20 mg/kg i.v. bolus and an infusion of 5 mg/kg/h i.v. for the duration of the experiment. Body temperature was maintained at 37°-39° C., and fluids infused were warmed to body temperature. The right carotid artery was exposed and dissected free of surrounding tissue, and a transonic flow probe of appropriate size was placed around the vessel. A Lexan constrictor (5 mm length) was placed onto the carotid artery. The constrictor was sized to abolish reactive hyperemia and to reduce mean carotid blood flow by no more than 50%-60%. Mechanical damage to the endothelium was induced in the constricted segment of the artery. Cyclic flow reductions due to platelet-dependent thrombus formation occurred shortly after placement of the constrictor over the region of endothelial damage. These gradual declines in carotid blood flow were occasionally interrupted spontaneously, but frequently required manual restoration by gently shaking the vessel. Flow reductions would typically return in 3-4 minutes. CFR frequency was calculated as the number of such cycles (CFRs) over a 30-minute period. On the basis of pilot studies, CFRs were evaluated in a maximum of four consecutive 30-minute collection periods, and mechanically mediated endothelial injury was performed at 30-minute intervals to ensure exposure of the thrombogenic endothelium.

Animals instrumented to produce CFRs were administered SCH 602539, cangrelor, or a combination of SCH 602539 and cangrelor. The dosing regimens of SCH 602539 were selected to provide a broad range of inhibition of platelet aggregation induced by 1 μM thrombin receptor agonist peptide (TRAP), whereas the doses of cangrelor were selected to achieve 50%-60% inhibition of platelet aggregation induced by 10 μM ADP (the degree of inhibition obtained with clopidogrel). Group 1 received drug vehicle only (20% hydroxypropyl betacyclodextran or saline, 2 mL i.v.). Group 2 received SCH 602539 dissolved in 20% hydroxypropyl betacyclodextran administered as sequential i.v. boluses of 0.1, 0.3, or 1 mg/kg, at 30-minute intervals. Group 3 received cangrelor dissolved in saline and administered as an intravenous infusion of 0.1, 0.2, or 0.3 μg/kg/min for 30 minutes each. Group 4 received a combination of SCH 602539 bolus plus cangrelor infusion (at respective doses of: 1) 0.05 mg/kg+0.05 μg/kg/min; 2) 0.1 mg/kg+0.1 μg/kg/min; and 3) 0.15 mg/kg+0.1 μg/kg/min. The three dose regimens within each group were administered sequentially over the consecutive 30-minute periods coinciding with the re-induction of endothelial injury. Study drug or vehicle was administered after stable CFRs were achieved, and upon the completion of a 30-minute baseline CFR collection period, drug(s) or vehicle were administered incrementally (every 30 minutes) either as a bolus (SCH 602539) or as an infusion (cangrelor), and their effects on CFRs were monitored in the next three 30-minute observation periods. Blood samples (3 mL) were collected from the femoral arterial catheter at the end of each 30-minute observation period for assessment of ex vivo platelet aggregation and coagulation parameters.

Platelet aggregation studies were performed ex viva on blood samples obtained from the monkeys subjected to the Folts model, using a ChronoLog whole blood aggregometer (Model 540VS, Havertown, Pa.). Briefly, 0.5 mL of blood was incubated with 0.5 mL of normal saline at 37° C. in a cuvette containing a stir bar for 2 minutes. Platelet agonists used in this study included TRAP (3 μM), ADP (10 μM), the thromboxane A₂ mimetic U46619 (10 μM), and collagen (3 μg/mL). Platelet aggregation was monitored for 5 minutes following the addition of the agonist. The peak aggregation response was recorded in ohms. In addition, standard coagulation parameters, including prothrombin time (PT), activated partial thromboplastin time (APTT), and activated clotting time (ACT), were assessed.

The in vivo effect of co-administration of PAR-1 and P2Y₁₂ antagonists on the inhibition of CFRs—synergy, additivity, or antagonism—was tested using criteria described by Berenbaum et al. (Berenbaum M C. Synergy, additivism and antagonism in immunosuppression. A critical review. Clin Exp Immunol. 1977; 28:1-18). Briefly, CFR frequency was plotted versus drug dose administered and fitted to dose-response curves using Inhibitory Effect Eₘₐₓ modeling. Estimates of the dose regimens needed for 50% reduction in CFRs (EC₅₀) were calculated using the equation, E=Eₘₐₓ (1−(C/(C+EC₅₀))), where E is the CFR frequency, assuming maximum frequency (Eₘₐₓ)) is achieved at dose level (C) 0 and zero frequency at dose level infinity. EC₅₀ a is 50% of maximum CFR frequency.

Dose-response curves were constructed for SCH 602539 and cangrelor. Equipotent dose values obtained for the tested combinations were fitted to the equation for each individual compound. These equipotent doses were then applied to the following equation used by Berenbaum for the determination of the synergy factor. Values less than 1 indicate the presence of synergy, whereas values equaling 1 are indicative of an additive effect.

$$\frac{Dose of {A}_{in combination with B}}{Dose of {A}_{Alone}} + \frac{Dose of {B}_{in combination with A}}{Dose of {B}_{Alone}}$$

Thus, the term “synergistic effect” as used herein, shall be understood to mean the consequence of a dosing regimen that results in a Berenbaum factor having a value of less than 1.

Of the 24 monkeys studied, 22 exhibited stable CFRs after instrumentation. There was minimal reduction in blood flow in the context of abolishing hyperemic blood flow. Heart rate, blood pressure, and body temperature were unchanged for the duration of the study.

Results of 22 experiments are shown in Table 1, which shows the in vivo results of the effects of SCH 602539, cangrelor, and their combination on CFRs and the ex vivo inhibition of platelet aggregation. The stability and utility of the surgical model was demonstrated by the consistent and reproducible CFRs achieved in animals treated with vehicle for the 2-hour study period. In the 22 experiments, the control frequency of the CFRs was 8.9+1.8 per 30 minutes, ranging from 7 to 13.

TABLE 1

| | | | |
|---|---|---|---|
| Baseline | Period 1 | Period 2 | Period 3 |

Control

| | | | | |
|---|---|---|---|---|
| CFRs | 8.6 + 0.6 | 6.6 + 0.8 | 8.4 + 0.4 | 8.8 + 0.7 |
| Aggrega- | | | | |
| tion | | | | |
| (ohms) | | | | |
| TRAP | 13 ± 3 | 12 ± 2 | 17 ± 4 | 14 ± 5 |
| ADP | 18 ± 2 | 17 ± 2 | 15 ± 1 | 16 ± 2 |
| U46619 | 21 ± 3 | 19 ± 2 | 21 ± 4 | 19 ± 4 |
| Collagen | 21 ± 3 | 24 ± 3 | 27 ± 4 | 29 ± 4 |
| SCH | Baseline | 0.1 mg/kg | 0.3 mg/kg | 1.0 mg/kg |
| 602539 | | | | |
| CFRs | 8.7 + 0.6 | 4.3 + 1.3 | 1.7 + 1.2 | 1.8 + 1.3 |
| Aggrega- | | | | |
| tion | | | | |
| (ohms) | | | | |
| TRAP | 14 ± 3 | 9 ± 4 | 2 ± 2 | 100 |
| ADP | 15 ± 3 | 14 ± 2 | 16 ± 3 | 13 ± 4 |
| U46619 | 20 ± 5 | 17 ± 3 | 19 ± 4 | 21 ± 5 |
| Collagen | 27 ± 4 | 26 ± 2 | 24 ± 3 | 29 ± 5 |
| Cangrelor | Baseline | 0.1 μg/kg/min | 0.2 μg/kg/min | 0.3 μg/kg/min |
| CFRs | 9.5 + 0.9 | 5.7 + 1.5 | 3.2 + 1.7 | 1.2 + 1.3 |
| Aggrega- | | | | |
| tion | | | | |
| (ohms) | | | | |
| TRAP | 13 ± 3 | 12 ± 3 | 12 ± 3 | 9 ± 3 |
| ADP | 14 ± 3 | 10 ± 2 | 7 ± 1 | 6 ± 1 |
| U46619 | 15 ± 3 | 12 ± 3 | 10 ± 4 | 10 ± 4 |
| Collagen | 24 ± 3 | 20 ± 3 | 20 ± 4 | 20 ± 3 |
| SCH | Baseline | 0.05 mg/kg + | 0.1 mg/kg + | 0.15 mg/kg + |
| 602539 + | | 0.05 μg/kg/min | 0.1 μg/kg/min | 0.1 μg/kg/min |
| Cangrelor | | | | |
| CFRs | 9.0 ± 1.3 | 2.4 ± 1.3 | 0.8 ± 0.9 | 0.6 ± 0.7 |
| Aggrega- | | | | |
| tion | | | | |
| (ohms) | | | | |
| TRAP | 23 ± 4 | 14 ± 3 | 10 ± 2 | 0 |
| ADP | 14 ± 1 | 10 ± 1 | 9 ± 2 | 7 ± 2 |
| U46619 | 22 ± 2 | 22 ± 3 | 15 ± 5 | 20 ± 3 |
| Collagen | 26 ± 3 | 30 ± 3 | 20 ± 6 | 22 ± 4 |

Intravenous bolus doses of SCH 602539 reduced the number of CFRs in a dose-dependent manner. CFR frequency was reduced from baseline by approximately 50% with the 0.1-mg/kg dose and by >80% with the 0.3- and 1.0-mg/kg doses (P<0.05 versus vehicle for both). CFRs were completely abolished in 4 of 6 animals with both the 0.3-mg/kg and 1.0-mg/kg doses.

Continuous infusions of cangrelor reduced CFR frequency from baseline by approximately 40%, 70%, and 90% with the 0.1-, 0.2-, and 0.3-μg/kg/min 30-minute infusion doses, respectively. The reductions achieved with the 2 highest doses were statistically significant versus vehicle. The intermediate and the highest doses of cangrelor completely prevented CFRs in 3/6 and 5/6 animals, respectively. No complete suppression was evident in any of the animals treated with the lowest dose of cangrelor.

In order to assess the antithrombotic effects of SCH 602539 in combination with cangrelor, doses of each agent that provided only modest inhibition of CFRs when used alone were chosen (SCH 602539: 0.1 mg/kg, and cangrelor: 0.1 μg/kg/min). Doses of SCH 602539 and cangrelor that were estimated not to impact CFR frequency when administered alone (SCH 602539: 0.05 mg/kg, and cangrelor: 0.05 μg/kg/min), as well as a slightly higher dose of SCH 602539 (0.15 mg/kg), were used to explore the possibility that the antithrombotic effects of a PAR-1 antagonist and a P2Y₁₂ ADP receptor antagonist were synergistic. Initial treatment with SCH 602539 0.05 mg/kg plus cangrelor 0.05 μg/kg/min significantly reduced the mean CFR frequency to 2.4 and completely abolished CFRs in 2 of 5 animals. Treatment with a combination of SCH 602539 0.1 mg/kg plus 0.1 μg/kg/min cangrelor completely abolished the CFRs in 4/5 animals and reduced the number of CFRs by approximately 50% in the remaining animal.

It is established that the Folts model of CFRs resulting from a fixed stenosis of an arterial bed is the result of the in vivo interplay of platelet-sensitive vasoactive molecules, which closely mimics the clinical scenario of unstable angina. EC₅₀ values calculated from dose-response curves are 0.10±0.042 mg/kg and 0.099±0.039 μg/kg/min for SCH 602539 and cangrelor, respectively.

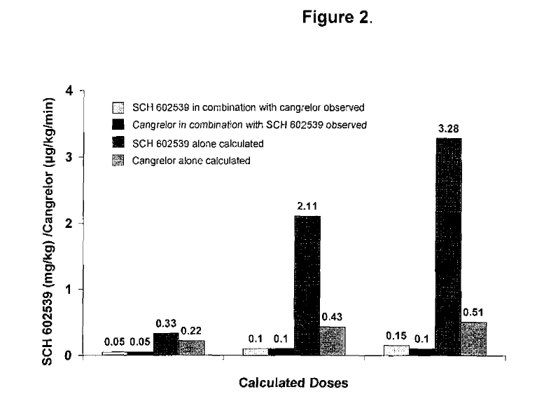

Five experiments were conducted for each of the three combinations partnering SCH 602539 and cangrelor. The corresponding equipotent doses for each compound compared with the different dose combinations were calculated from the respective dose-response curve (FIG. 2). The in vivo CFR inhibition exerted by SCH 602539 or cangrelor when combined with its partner was greater than that of the individual components administered alone.

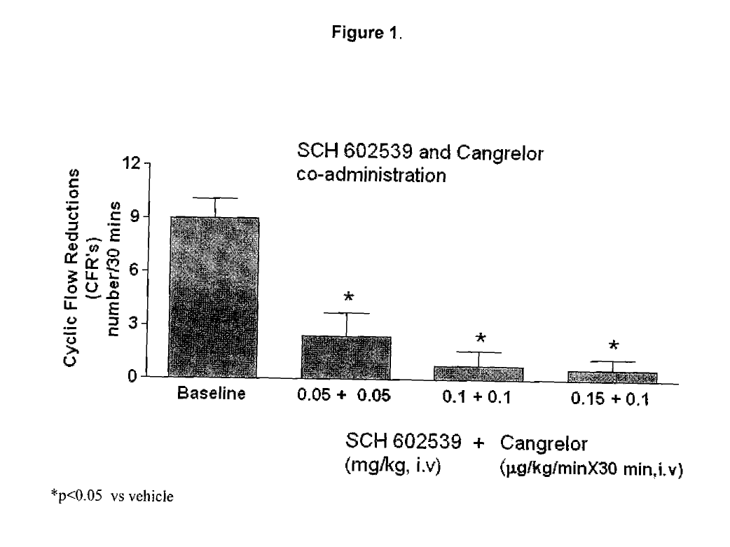

The presence of a synergistic interaction is suggested by the 50% reduction in CFR frequency with SCH 602539 0.05 mg/kg in combination with cangrelor 0.05 μg/kg/min and the complete extinction of CFR production in 4 of 5 animals receiving SCH 602539 0.1 mg/kg plus cangrelor 0.1 μg/kg/min. Similar antithrombotic effects were also observed with the combination of SCH 602539 0.15 mg/kg plus cangrelor 0.1 μg/kg/min administered in the subsequent 30-minute period. The CFR frequency for baseline and each of the 3 doses administered, along with the calculated doses, is shown for SCH 602539 and cangrelor in FIGS. 1 and 2.

Synergism was confirmed, as the synergistic factor was less than 1 in 12 of 15 combination experiments and was seen in the lowest tested dosage combination of 0.05 plus 0.05. Using the Berenbaum model, the calculated mean synergistic factor for all 15 combination experiments was 0.41±0.17.

The effects of SCH 602539 alone, cangrelor alone, and the combination of SCH 602539 and cangrelor on ex vivo platelet aggregation mediated by various agonists (e.g., TRAP, ADP, thromboxane A₂ mimetic U46619, and collagen) in the Folts model of thrombosis in anesthetized cynomolgus monkeys were also evaluated. Table 1 outlines the inhibition of platelet aggregation noted at each of the tested dosing regimens of study drug. The two highest doses of SCH 602539 (0.3 mg/kg and 1 mg/kg) were associated with potent (>80%) and dose-related inhibition of ex vivo platelet aggregation induced by 3 μM TRAP, but did not affect the aggregation induced by 10 μM ADP, 10 μM thromboxane A₂ mimetic U46619, and 3 μg/mL collagen, demonstrating selectivity of SCH 602539 for the PAR-1 receptor pathway. The two highest doses of cangrelor (0.2 and 0.3 μg/kg/min) inhibited ADP-mediated platelet aggregation by approximately 50% to 60% (as targeted) but did not interfere with the aggregation stimulated by 3 μM TRAP, 10 μM thromboxane A₂ mimetic U46619, or 3 μg/mL collagen. Finally, the combination of SCH 602539 and cangrelor inhibited the aggregation induced by 3 μM TRAP and 10 μM ADP in a dose-related manner, and the level of inhibition was the same as for each agent used alone. No inhibition of platelet aggregation induced by 10 μM thromboxane A₂ mimetic U46619 or 3 μg/mL collagen was observed. These findings suggest that the ex vivo inhibition of TRAP- and ADP-mediated platelet aggregation pathways are not predictive of the synergistic effect of combined therapy with SCH 602539 and cangrelor on the reduction of CFRs.

Effects of vehicle, SCH 602539, cangrelor, and the combination of SCH 602539 plus cangrelor on prothrombin time (“PT”), activated partial thromboplastin time (“APTT”), and activated clotting time (“ACT”) in the Folts model of thrombosis in anesthetized cynomolgus monkeys are displayed in Table 2. PT and APTT assays were performed with plasma, while the ACT assay was performed with whole blood. Data are presented as mean±SEM (n=5-6/group).

Administration of SCH 602539 alone, cangrelor alone, and the combination of SCH 602539 and cangrelor had no effect on the coagulation parameters, including PT, APTT, and ACT. These findings are consistent with the fact that both SCH 602539 and cangrelor are antiplatelet agents that interact with specific platelet receptors, and do not interfere with the activity of the coagulation cascade.

TABLE 2

| Coagulation | | 20% Hydroxypropyl Betacyclodextran | | |
|---|---|---|---|---|
| Coagulation | | Cangrelor (μg/kg/min × 30 min, i.v.) | | |
| Parameter | Baseline | Vehicle | Vehicle | Vehicle |
| Parameter | Baseline | 0.1 | 0.2 | 0.3 |
| PT (sec) | 11.2 ± 0.4 | 11.4 ± 0.2 | 11.3 ± 0.6 | 12.4 ± 0.6 |
| PT (sec) | 10.6 ± 0.4 | 10.4 ± 0.3 | 10.8 ± 0.6 | 10.7 ± 0.6 |
| APTT (sec) | 24.6 ± 5.4 | 23.9 ± 3.8 | 22.9 ± 3.1 | 29.9 ± 10.2 |
| APTT (sec) | 22.5 ± 1.6 | 23.6 ± 2.6 | 22.1 ± 1.3 | 22.2 ± 2.0 |
| ACT (sec) | 75 ± 2.3 | 69 ± 1.8 | 67 ± 3.8 | 69 ± 4 |
| ACT (sec) | 77 ± 3.7 | 79 ± 2.3 | 76 ± 1.7 | 74 ± 3.5 |
| | | SCH 602539 (mg/kg, i.v., bolus) | | |
| Coagulation | | SCH 602539 (mg/kg, i.v., bolus) + | | |
| Parameter | | Cangrelor (μg/kg/min × 30 min, i.v.) | | |
| Coagulation | Baseline | 0.1 | 0.3 | 1.0 |
| PT (sec) | 11.6 ± 0.4 | 11.5 ± 0.2 | 11.8 ± 0.4 | 12.8 ± 1.1 |
| Parameter | Baseline | 0.05 + 0.05 | 0.1 + 0.1 | 0.15 + 0.15 |
| APTT (sec) | 22.8 ± 1.7 | 22.4 ± 1.5 | 23.4 ± 2.2 | 25.4 ± 4.9 |
| PT (sec) | 10.4 ± 0.3 | 10.5 ± 0.3 | 10.3 ± 0.2 | 10.6 ± 0.3 |
| ACT (sec) | 79 ± 5.9 | 73 ± 2.2 | 71 ± 4.4 | 67 ± 4.9 |
| APTT (sec) | 24.7 ± 2.2 | 27.3 ± 2.8 | 23.8 ± 2.2 | 26.8 ± 2.8 |
| ACT (sec) | 77 ± 8.2 | 81 ± 3.7 | 87 ± 3.4 | 83 ± 5.1 |

In the above-described study, the inventors demonstrated the antithrombotic effects of PAR-1 antagonism with SCH 602539 in a Folts model of thrombosis. Furthermore, they have demonstrated that the in vivo antithrombotic effects of PAR-1 antagonism in combination with P2Y₁₂ ADP receptor antagonism are synergistic. The inhibitory activity of SCH 602539 was specific for platelet aggregation induced by TRAP, whereas the aggregation induced by other agonists, such as ADP, thromboxane A₂ mimetic 046619, and collagen, was not affected, demonstrating the specificity and selectivity for the PAR-1 receptor. Cangrelor specifically inhibited aggregation induced by ADP and demonstrated a modest numeric inhibition of aggregation induced by collagen, but not by TRAP or U46619. These findings parallel those reported in the literature. (Eikelboom J W, Hankey G J, Thom J, Claxton A, Yi Q, Gilmore G, Staton J, Barden A, Norman P E. Enhanced antiplatelet effect of clopidogrel in patients whose platelets are least inhibited by aspirin: a randomized crossover trial. J Thromb Haemost. 2005; 3:2649-2655. Storey R F, Wilcox R G, Heptinstall S. Comparison of the pharmacodynamic effects of the platelet ADP receptor antagonists clopidogrel and AR-C69931 MX in patients with ischaemic heart disease. Platelets. 2002; 13:407-413).

As expected, neither SCH 602539 nor cangrelor (nor the combination of the 2) had any notable effect on coagulation parameters, a finding that is consistent with the fact that these agents interact with specific platelet receptors and do not interfere with the coagulation cascade.

Synergistic antithrombotic effects similar to those observed with the combination of SCH 602539 and cangrelor in this study have been reported with other combinations of antiplatelet agents (Bierbach B, Horstick G, Berg O, Heimann A, Munzel T, Vahl C F, Kempski O, Darius H. Potent low dose platelet inhibitory effects of clopidogrel and aspirin on coronary thrombus formation in an animal model of acute unstable angina. Thromb Haemost. 2006; 95:354-361. Herbert J M, Dol F, Bernat A, Falotico R, Lalé A, Savi P. The antiaggregating and antithrombotic activity of clopidogrel is potentiated by aspirin in several experimental models in the rabbit. Thromb Haemost. 1998; 80:512-518), that target distinct platelet activation pathways contributing to thrombosis. In the Folts model of thrombosis in pigs, the combination of low oral doses of clopidogrel (0.1 mg/kg) and aspirin (1 mg/kg) completely eliminated CFRs at 90 minutes, whereas the higher doses of each agent alone (clopidogrel 5 mg/kg and aspirin 7 mg/kg) reduced, but did not completely abolish, the CFRs. (Bierbach B, Horstick G, Berg O, Heimann A, Munzel T, Vahl C F, Kempski O, Darius H. Potent low dose platelet inhibitory effects of clopidogrel and aspirin on coronary thrombus formation in an animal model of acute unstable angina. Thromb Haemost. 2006; 95:354-361). A separate study in rabbits that employed several different models of thrombosis demonstrated that the addition of oral aspirin to oral clopidogrel was associated with potent antithrombotic effects, but also additive bleeding effects, possibly related to the combined inhibitory activity of 2 antiplatelet agents on collagen-induced platelet aggregation. (Herbert J M, Dol F, Bernat A, Falotico R, Lalé A, Savi P. The antiaggregating and antithrombotic activity of clopidogrel is potentiated by aspirin in several experimental models in the rabbit. Thromb Haemost. 1998; 80:512-518). Additive antithrombotic effects of combined inhibition of the thromboxane A₂ and P2Y₁₂ ADP receptor platelet activation pathways with aspirin and clopidogrel observed in these studies are consistent with significant reductions in ischemic events with dual antiplatelet therapy over aspirin alone reported in large clinical trials. (Wiviott S D, Braunwald E, McCabe C H, Montalescot G, Ruzyllo W, Gottlieb S, Neumann F J, Ardissino D, De Servi S, Murphy S A, Riesmeyer J, Weerakkody G, Gibson C M, Antman E M; TRITON-TIMI 38 Investigators. Prasugrel versus clopidogrel in patients with acute coronary syndromes. N Engl J Med. 2007; 357:2001-2015. Yusuf S, Zhao F, Mehta S R, Chrolavicius S, Tognoni G, Fox K K; Clopidogrel in Unstable Angina to Prevent Recurrent Events Trial Investigators. Effects of clopidogrel in addition to aspirin in patients with acute coronary syndromes without ST-segment elevation. N Engl J Med. 2001; 345:494-502. Chen Z M, Jiang L X, Chen Y P, Xie J X, Pan H C, Peto R, Collins R, Liu L S; COMMIT (Clopidogrel and Metoprolol in Myocardial Infarction Trial) collaborative group. Addition of clopidogrel to aspirin in 45,852 patients with acute myocardial infarction: randomised placebo-controlled trial. Lancet. 2005; 366:1607-1621. Sabatine M S, Cannon C P, Gibson C M, López-Sendón J L, Montalescot G, Theroux P, Claeys M J, Cools F, Hill K A, Skene A M, McCabe C H, Braunwald E; CLARITY-TIMI 28 Investigators. Addition of clopidogrel to aspirin and fibrinolytic therapy for myocardial infarction with ST-segment elevation. N Engl J Med. 2005; 352:1179-1189. Steinhubl S R, Berger P B, Mann J T III, Fry E T, DeLago A, Wilmer C, Topol E J; CREDO Investigators. Clopidogrel for the Reduction of Events During Observation. Early and sustained dual oral antiplatelet therapy following percutaneous coronary intervention: a randomized controlled trial. JAMA. 2002; 288:2411-2420). These findings suggest that in the vascular bed, platelet activation leading to thrombosis is a complex matrix mediated by multiple pathways.

By these results, the inventors have demonstrated the in vivo synergism with the combination of a PAR-1 antagonist and a P2Y₁₂ ADP receptor antagonist. Whereas the binding of thrombin to the PAR-1 receptor represents one of the most potent platelet activation pathways leading to thrombosis, neither aspirin nor P2Y₁₂ ADP receptor antagonists (including cangrelor) significantly inhibit the PAR-1 pathway. The presence of a synergistic effect resulting from the combined administration of a P2Y₁₂ antagonist with the direct thrombin inhibitor melagatran has been previously described. (Nylander S, Mattsson C, Ramström S, Lindahl T L. Synergistic action between inhibition of P2Y12/P2Y1 and P2Y12/thrombin in ADP- and thrombin-induced human platelet activation. Br J Pharmacol. 2004; 142:1325-1331).

There are two possible explanations for this synergism. The first is via the concurrent inhibition of the G protein-coupled receptor G_{αq}, which mediates both P2Y₁ and PAR-1 transmembrane signaling. G_{αq} and G_{αi} are the principal secondary intracellular signals for ADP and are localized to the P2Y₁ and P2Y₁₂ receptors, respectively. (Jin J, Kunapuli S P. Coactivation of two different G protein-coupled receptors is essential for ADP-induced platelet aggregation. Proc Natl Acad Sci USA. 1998; 95:8070-8074). G_{αq} is the more potent of the 2 G protein-coupled receptors. Thrombin-mediated platelet activation requires G_{αq} localized to the PAR-1 and PAR-4 transmembrane receptors. (Coughlin S R. Protease-activated receptors in hemostasis, thrombosis and vascular biology. J Thromb Haemost. 2005; 3:1800-1814). It is hypothesized that blockade of PAR-1 will directly block G_{αq} signaling, rendering the platelet incapable of reacting to either thrombin or P2Y₁-mediated ADP stimulation. However, G_{αq} receptor responsiveness itself is not impacted by the direct inhibition of the transmembrane PAR-1 or P2Y₁ receptors, making it less likely that such an intracellular phenomenon occurs. The second possible mechanism relies on the platelet's paracrine effect on ADP secretion and the signal amplification mechanism resulting from such secretion. Thrombin, the most potent platelet agonist, activates platelets directly by phospholipase C-mediated calcium release and Rho-mediated platelet shape change. However, thrombin also generates activation amplification through the secondary release of ADP from the dense granules. Blocking thrombin-mediated platelet secretion may result in a reduction in the total ADP pool available to the localized site. This would result in less agonist/antagonist competition for P2Y₁₂ receptors and a greater level of inhibition of ADP-mediated platelet aggregation. Such triple inhibition may allow for lower levels of ADP receptor blockade using currently available agents while increasing efficacy and reducing risk of bleeding. Additionally, triple therapy may obviate the current perceived clinical need for potent ADP receptor antagonists.

The challenges clinically are to determine whether the addition of a PAR-1 antagonist to the standard of care of aspirin and a thienopyridine will provide incremental clinical benefit without incremental bleeding risk, and in doing so what will be the optimal dose for each agent. The combined inhibition of thromboxane A₂ and P2Y₁₂ ADP receptor platelet activation pathways with aspirin and a thienopyridine provides more potent antithrombotic activity than either agent alone and has been documented to reduce the rate of ischemic outcomes compared with aspirin alone. (Wiviott S D, Braunwald E, McCabe C H, Montalescot G, Ruzyllo W, Gottlieb S, Neumann F J, Ardissino D, De Servi S, Murphy S A, Riesmeyer J, Weerakkody G, Gibson C M, Antman E M; TRITON-TIMI 38 Investigators. Prasugrel versus clopidogrel in patients with acute coronary syndromes. N Engl J Med. 2007; 357:2001-2015. Yusuf S, Zhao F, Mehta S R, Chrolavicius S, Tognoni G, Fox K K; Clopidogrel in Unstable Angina to Prevent Recurrent Events Trial Investigators. Effects of clopidogrel in addition to aspirin in patients with acute coronary syndromes without ST-segment elevation. N Engl J Med. 2001; 345:494-502). Recent studies of a more potent thienopyridine, prasugrel, resulted in an incremental 19% reduction in clinical events, but there remains a 10% prevalence of clinical events complexed with a 32% increased bleeding risk, including major hemorrhage during coronary artery bypass grafting and intracranial hemorrhage during stroke. (Wiviott S D, Braunwald E, McCabe C H, Montalescot G, Ruzyllo W, Gottlieb S, Neumann F J, Ardissino D, De Servi S, Murphy S A, Riesmeyer J, Weerakkody G, Gibson C M, Antman E M; TRITON-TIMI 38 Investigators. Prasugrel versus clopidogrel in patients with acute coronary syndromes. N Engl J Med. 2007; 357:2001-2015). A conceptual therapeutic window model of P2Y₁₂ inhibition has been proposed by Gurbel (Gurbel P A, Tantry U S. Selecting optimal antiplatelet therapy based on platelet function monitoring in patients with coronary artery disease. Curr Treat Options Cardiovasc Med. 2009; 11:22-32), that defines the delicate balance between efficacy and bleeding. Such a model would establish a warfarin International Normalized Ratio (INR)-like range that factors the type of ADP inhibition and the presence of PAR-1 inhibition. These data would suggest that in the context of the Gurbel model, PAR-1 inhibition would improve therapeutic outcomes, widen the therapeutic range of ADP inhibition, and reduce ADP-mediated risk of bleeding by allowing lower levels of ADP-mediated inhibition of platelet aggregation.

In conclusion, the inventors have surprisingly demonstrated that combined P2Y₁₂ and PAR-1 antagonism can result in the synergistic inhibition of CFRs in the Folts model of fixed arterial stenosis, suggesting the possible clinical benefit of blocking multiple platelet pathways. Such a concept is being tested in the ongoing phase 3 megatrials of SCH 530348. The proven synergistic effect constitutes evidence of a surprising benefit of co-administration of a thrombin receptor antagonist selective for PAR-1 antagonism and a P2Y₁₂ ADP receptor antagonist, and specifically of the combination of SCH 602539 and cangrelor.

The present invention is directed to the use of any combination of any thrombin receptor antagonist and any P2Y₁₂ADP receptor antagonist for the treatment of thrombosis or any condition that could benefit from the blocking of multiple platelet activation pathways. Thus, the invention includes the use of thrombin receptor antagonists described in U.S. Pat. Nos. 7,304,078, including SCH 530348, and 6,645,987 and 7,488,742, including SCH 590709, whose structural formula is as follows:

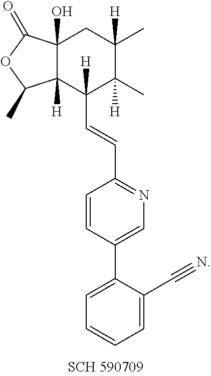

Also included among the combinations within the present invention are those in which the thrombin receptor antagonist is E5555 currently in development by Eisai:

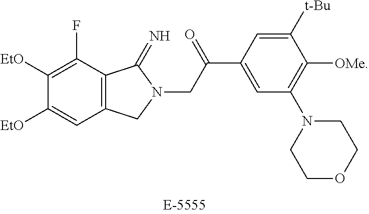

Various embodiments of the invention include the various free base and salt forms of the PAR-1 and of a P2Y₁₂ADP receptor antagonist components, and combinations of such free base and salt forms. Regarding PAR-1 receptor antagonists, the free base form of SCH 602539 was administered in the in vivo experiments described above. The bisulfate salt of SCH 530348 is currently in clinical trials, and embodiments including the free base form are also preferred. Regarding P2Y₁₂ ADP receptor antagonists, the bisulfate form of clopidogrel is the active in Plavix®.

The compounds that are PAR-1 and of a P2Y₁₂ ADP receptor antagonists can form salts, which are also within the scope of this invention. Reference to one of these compounds herein is generally understood to include reference to salts and free bases thereof, unless otherwise indicated. The term “salt(s),” as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds may be formed, for example, by reacting a compound free base with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates (and bisulfates), tartarates, thiocyanates, toluenesulfonates (also known as tosylates,) and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (e.g., organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g. dimethyl, diethyl, and dibutyl sulfates), long chain halides (e.g., decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The combinations of the present invention have antithrombotic effect, and will be useful in prevention and treatment of diseases and conditions in which such antithrombotic effect is particularly desirable. Such diseases and conditions include without limitation acute coronary syndrome, thrombosis, stroke, myocardial infarction, secondary prevention, prevention of thrombotic events in patients who have undergone percutaneous coronary intervention or cardiopulmonary bypass surgery (“CPB”), including coronary artery bypass surgery (“CABG”), cardiac valvular repair and replacement surgery, pericardial and aortic repair surgeries.

“Acute coronary syndrome” includes any group of clinical symptoms compatible with acute myocardial ischemia. Acute myocardial ischemia is chest pain due to insufficient blood supply to the heart muscle that results from coronary artery disease (also called coronary heart disease). Acute coronary syndrome thus covers the spectrum of clinical conditions ranging from unstable angina to non-Q-wave myocardial infarction and Q-wave myocardial infarction. Symptoms may include chest pain, shortness of breath, nausea, vomiting, diaphoresis (sweating), palpitations, anxiety or a sense of impending doom and a feeling of being acutely ill.

“Secondary prevention” refers to the treatment of patients who have already suffered a significant cardiovascular event, such as a heart attack or stroke, to prevent another future, potentially more serious, perhaps lethal, cardiovascular or cerebrovascular event.

Another cardiovascular condition for which the inventive combinations may be useful is peripheral arterial disease (“PAD”), also known as peripheral vascular disease (“PVD”), which occurs when cholesterol and scar tissue build up, forming plaque inside the arteries that narrows and clogs the arteries. The clogged arteries cause decreased blood flow to the legs, which can result in pain when walking, and eventually gangrene and amputation.

The present invention includes co-administration of the thrombin receptor antagonist and the P2Y₁₂ADP receptor antagonist either as separate dosage forms or as a single dosage form. Each component is administered in a therapeutically effective amount, i.e., the amount of the thrombin receptor antagonist is effective in antagonism of a thrombin receptor and the amount of the P2Y₁₂ADP receptor antagonist is effective in antagonism of a P2Y₁₂ADP receptor, thus producing the desired therapeutic, ameliorative, inhibitory or preventative effect by blocking of multiple platelet activation pathways.

P2Y₁₂ADP receptor antagonists ticlopidine and clopidogrel are structurally related compounds, belonging to the thienopyridine family of ADP receptor antagonists; they are pro-drugs that are inactive in vitro and need to be metabolized in vivo by the hepatic cytochrome P-450 1A enzymatic pathway to active metabolites, which have very short half-lives. Prasugrel is a newer thienopyridine compound with a much faster onset of action than clopidogrel. (Niitsu Y, Jakubowski J A, Sugidachi A, Asai F. Pharmacology of CS-747 (Prasugrel, LY640315), a novel, potent antiplatelet agent with in vivo P2Y₁₂ receptor antagonist activity. Semin Thromb Hemost 2005; 31: 184-194.) Cangrelor is a selective and reversible direct inhibitor of P2Y₁₂. (Husted S, Emanuelsson H, Heptinstall S, Sandset P M, Wickens M, Peters G. Pharmacodynamics, pharmacokinetics, and safety of the oral reversible P2Y₁₂ antagonist AZD6140 with aspirin in patients with atherosclerosis: a double-blind comparison to clopidogrel with aspirin. Eur Heart J 2006; 27: 1038-1047. First published on Feb. 13, 2006, doi:10.1093/eurheartj/ehi754.) AZD6140 (a compound in development by AstraZeneca) is the first oral reversible P2Y₁₂ antagonist. (Husted et al.) ARC109318, is the first selective and stable, non-phosphate, competitive P2Y₁₂ antagonist. (van Giezen J J J, Humphries R G. Preclinical and clinical studies with selective reversible direct P2Y12 antagonists. Semin Thromb Hemost 2005; 31: 195-204.) PRT060128 (a compound in development by Portola Pharmaceuticals) is the only reversible, direct acting, IV and oral ADP receptor antagonist in clinical development. All of these agents, as well as any other agents having activity as P2Y₁₂ADP receptor antagonists, are within the scope of the invention as the P2Y₁₂ ADP receptor antagonist component of the therapeutic combination.

The invention further encompasses pharmaceutical compositions comprising therapeutically effective amounts of a thrombin receptor antagonist selective for PAR-1 and of a P2Y₁₂ADP receptor antagonist. The pharmaceutical compositions may be in the form of an oral tablet, capsule or syrup, or a parenteral formulation. As described above, various embodiments of the invention include pharmaceutical compositions comprising various salt forms or free bases of the PAR-1 and of the P2Y₁₂ADP receptor antagonist components, and the various combinations thereof.

The invention further encompasses kits comprising pharmaceutical compositions of each of a thrombin receptor antagonist selective for PAR-1 and of a P2Y₁₂ADP receptor antagonist. The forms of the pharmaceutical compositions in the kit may be the same (e.g., both parenteral) or they may differ (e.g., one an oral tablet, the other parenteral).

It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

We claim:

1. A method of treating or preventing a cardiovascular condition comprising administering to a patient in need of such treatment or prevention therapeutically effective amounts of a thrombin receptor antagonist selective for PAR-1 and of a P2Y₁₂ ADP receptor antagonist.

2. The method according to claim 1, wherein the thrombin receptor antagonist is a compound having the chemical formula: 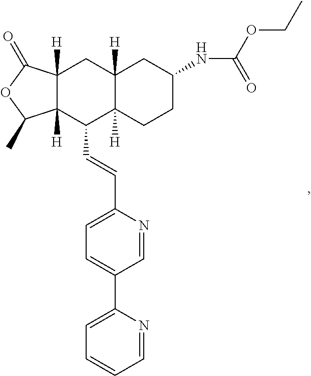
in the form of the free base, or of a pharmaceutically acceptable salt.

3. The method according to claim 1, wherein the thrombin receptor antagonist is a compound having the chemical formula: 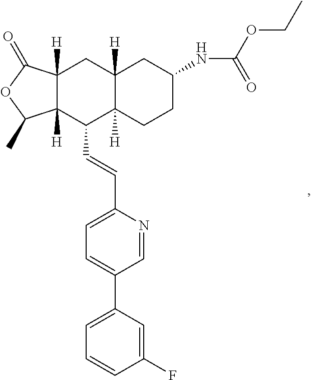
in the form of the free base, or of a pharmaceutically acceptable salt.

4. The method according to claim 1, wherein the thrombin receptor antagonist is a compound having the chemical formula: 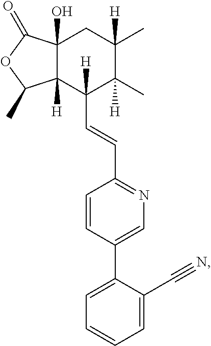
in the form of the free base, or of a pharmaceutically acceptable salt.

5. The method according to claim 1, wherein the thrombin receptor antagonist is a compound having the chemical formula: 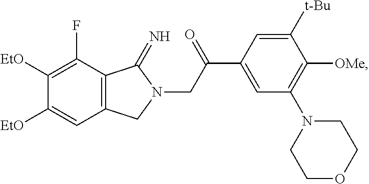
in the form of the free base, or of a pharmaceutically acceptable salt.

6. The method according to claim 2, wherein the P2Y₁₂ADP receptor antagonist is cangrelor, in the form of the free base, or of a pharmaceutically acceptable salt.

7. The method according to claim 1 wherein the P2Y₁₂ADP receptor antagonist is selected from the group consisting of ticlopidine, clopidogrel, AZD6140, ARC109318, and PRT060128, in the form of the free base, or of a pharmaceutically acceptable salt.

8. The method according to claim 1, wherein the cardiovascular condition is selected from the group consisting of acute coronary syndrome, thrombosis, stroke, myocardial infarction, peripheral arterial disease, thrombotic events in patients who have undergone percutaneous coronary intervention or cardiopulmonary bypass surgery, including coronary artery bypass surgery, cardiac valvular repair and replacement surgery, pericardial and aortic repair surgery.

9. The method according to claim 1, wherein the prevention is secondary prevention.

10. The method according to claim 1, wherein the thrombin receptor antagonist, the P2Y₁₂ ADP receptor antagonist, and the amounts of each are selected to generate a synergistic effect in the patient.

11. A pharmaceutical composition comprising therapeutically effective amounts of a thrombin receptor antagonist selective for PAR-1 and of a P2Y₁₂ ADP receptor antagonist, wherein the thrombin receptor antagonist, the P2Y₁₂ ADP receptor antagonist, and the amounts of each are selected to generate a synergistic effect in the patient.

12. The pharmaceutical composition according to claim 11, wherein the thrombin receptor antagonist is a compound selected from those having the chemical formulae: 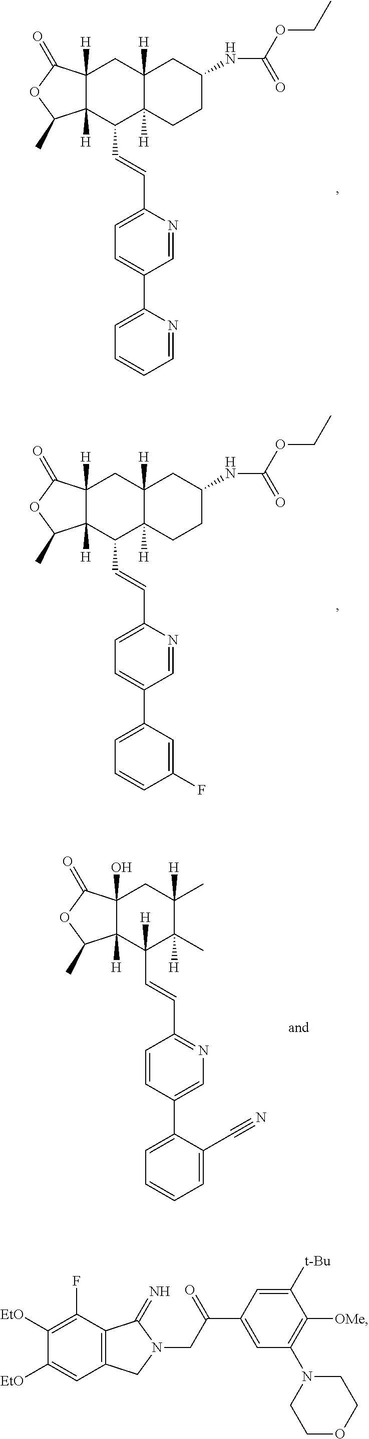
and wherein the P2Y₁₂ADP receptor antagonist is selected from the group consisting of cangrelor, ticlopidine, clopidogrel, AZD6140, ARC109318, and PRT060128, and wherein both the thrombin receptor antagonist and the P2Y₁₂ ADP receptor antagonist are independently in the form of the free base, or of a pharmaceutically acceptable salt.

13. A kit comprising a first pharmaceutical composition comprising a therapeutically effective amount of a thrombin receptor antagonist selective for PAR-1 and a second pharmaceutical composition comprising a therapeutically effective amount of a P2Y₁₂ ADP receptor antagonist, wherein the thrombin receptor antagonist, the P2Y₁₂ADP receptor antagonist, and the amounts of each are selected to generate a synergistic effect in the patient.

14. The kit according to claim 13, wherein the thrombin receptor antagonist is a compound selected from those having the chemical formulae: 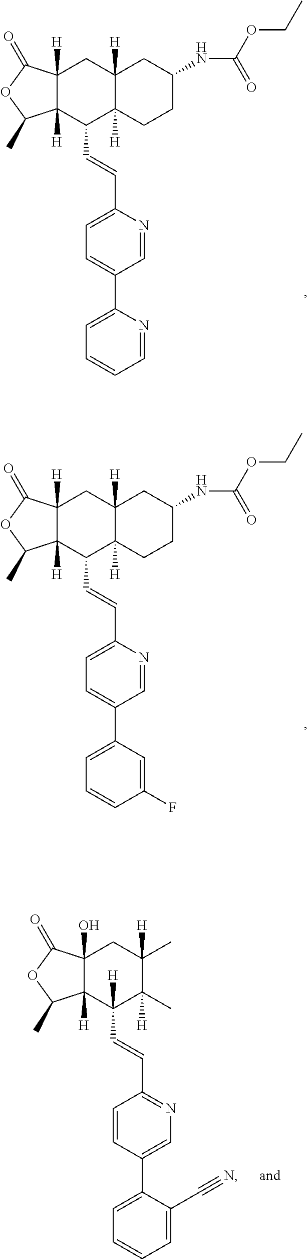 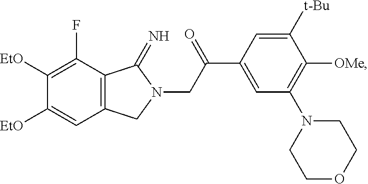
and wherein the P2Y₁₂ ADP receptor antagonist is selected from the group consisting of cangrelor, ticlopidine, clopidogrel, AZD6140, ARC109318, and PRT060128, wherein both the thrombin receptor antagonist and the P2Y₁₂ADP receptor antagonist are independently in the form of the free base, or of a pharmaceutically acceptable salt.
